(19) United States
(12) Patent Application Publication
(10) Pub. No.: US 2013/0090624 A1
(43) Pub. Date: Apr. 11, 2013

(54) RECAPTURE SHEATH

(71) Applicant: Boston Scientific Scimed, Inc., Maple Grove, MN (US)

(72) Inventor: Joel R. Munsinger, Blaine, MN (US)

(73) Assignee: BOSTON SCIENTIFIC SCIMED, INC., Maple Grove, MN (US)

(21) Appl. No.: 13/646,474

(22) Filed: Oct. 5, 2012

Related U.S. Application Data
(60) Provisional application No. 61/545,896, filed on Oct. 11, 2011.

(51) Int. Cl. A61M 25/10

(52) U.S. Cl. 604/500; 604/103.05

(57) ABSTRACT

In some embodiments, a recapturing sheath comprises an elongate shaft having a proximal end and a distal end. The shaft defines a lumen therein. The distal end comprises a flare and a slit. A first portion of said flare overlaps a second portion of said flare.

### CROSS-REFERENCE TO RELATED APPLICATIONS

This application claims the benefit of U.S. Provisional Application No. 61/545,896, filed Oct. 11, 2011, the entire content of which is hereby incorporated herein by reference.

### STATEMENT REGARDING FEDERALLY SPONSORED RESEARCH

Not Applicable

### BACKGROUND OF THE INVENTION

This invention relates generally to medical devices and more specifically to intravascular devices capable of delivering a drug to a localized area.

Balloon catheters are known in the art and are used for a variety of procedures, such as angioplasty and stenting. In some cases, balloon catheters are used to deliver drugs to tissue at a desired location of a body lumen or cavity. Typically, the balloon becomes positioned at the desired location for drug delivery by delivering it through portions of the body lumen or cavity where no drug delivery is desired. It is desirable to provide drug exposure at a target site while minimizing any drug exposure to non-target locations.

When a balloon is positioned in a bodily lumen that transports fluids, such as a blood vessel, the fluids are capable of carrying drug away from the treatment site. Further, upon balloon deflation, the balloon can assume a shape that causes difficulties in removal while avoiding drug loss. For example, a balloon can be delivered to a target site in a guide catheter and then be advanced out of the guide catheter for inflation and drug delivery. Upon deflation, the balloon might assume a flattened shape. The flattened balloon may span a dimension that exceeds the diameter of the guide catheter. As the flattened balloon is withdrawn back into the guide catheter, contact between the balloon and edges of the guide catheter can cause drug loss, such as drug coating being scraped off the balloon surface.

U.S. Pat. No. 5,954,706 teaches an example of a balloon suitable for drug delivery.

There remains a need for medical device designs arranged to contain drugs before, during and after delivery of the drug to a target site.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In some embodiments, a recapturing sheath comprises an elongate shaft having a proximal end and a distal end. The shaft defines a lumen therein. The distal end comprises a flare and a slit. A first portion of said flare overlaps a second portion of said flare.

In some embodiments, a medical assembly comprises a drug delivering medical device and a recapturing device. The recapturing device comprises an elongate shaft portion and a distal sheath portion. The distal sheath portion comprises a flared distal end. The recapturing device includes an internal lumen, and the medical device is oriented within the lumen. In some embodiments, an insertion sheath surrounds at least a distal portion of the recapturing device.

In some embodiments, a method comprises providing a drug delivering medical device and a recapturing device, the recapturing device having a flared end portion. The medical device and the recapturing device are delivered to a treatment site. If necessary, the flared end portion is oriented in a flared configuration. The drug is delivered to the treatment site, while at least some of said drug is contained in the recapturing device.

These and other embodiments which characterize the invention are pointed out with particularity in the claims annexed hereto and forming a part hereof. However, for a better understanding of the invention, its advantages and objectives obtained by its use, reference can be made to the drawings which form a further part hereof and the accompanying descriptive matter, in which there are illustrated and described various embodiments of the invention.

### BRIEF DESCRIPTION OF THE DRAWINGS

A detailed description of the invention is hereafter described with specific reference being made to the drawings.

FIG. 1 is a cross-sectional side view of an embodiment of a recapturing device.

FIGS. 2 and 3 show an embodiment of an insertion sheath.

FIG. 4 shows a cross-sectional side view of another embodiment of a recapturing device.

FIGS. 5 and 6 show embodiments of seats for a medical device.

FIG. 7 shows a slit distal end of a recapturing sheath.

FIGS. 8 and 9 show a slit distal end of a recapturing sheath at various sizes, having different degrees of overlap.

FIG. 10 shows an embodiment of a recapturing sheath within a body vessel, in use with a drug delivering balloon.

FIG. 11 shows another embodiment of a flared end of a recapturing sheath.

FIGS. 12 and 13 show embodiments of a folded distal end of a recapturing sheath.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

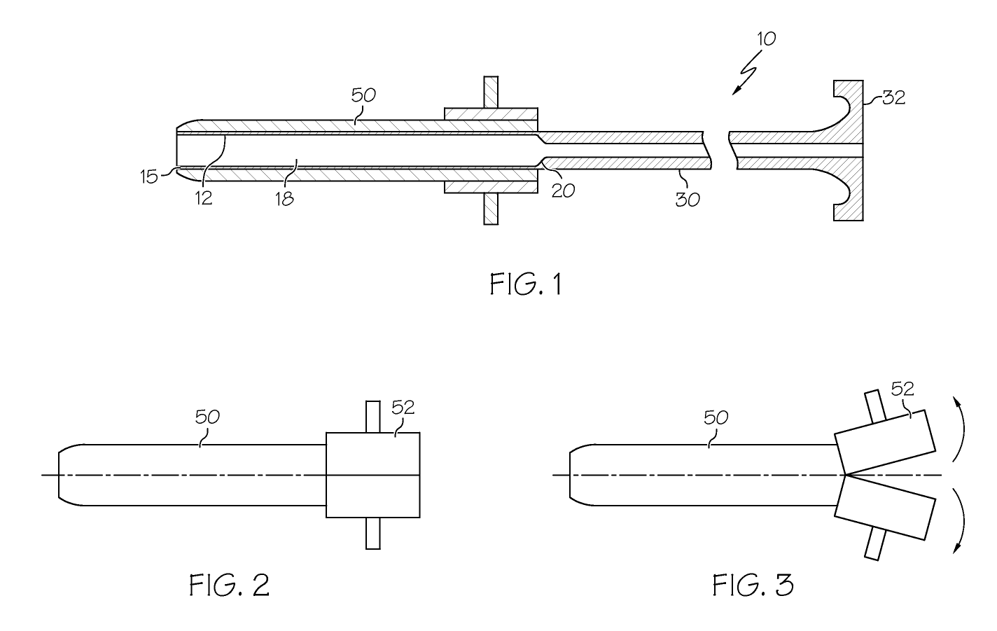

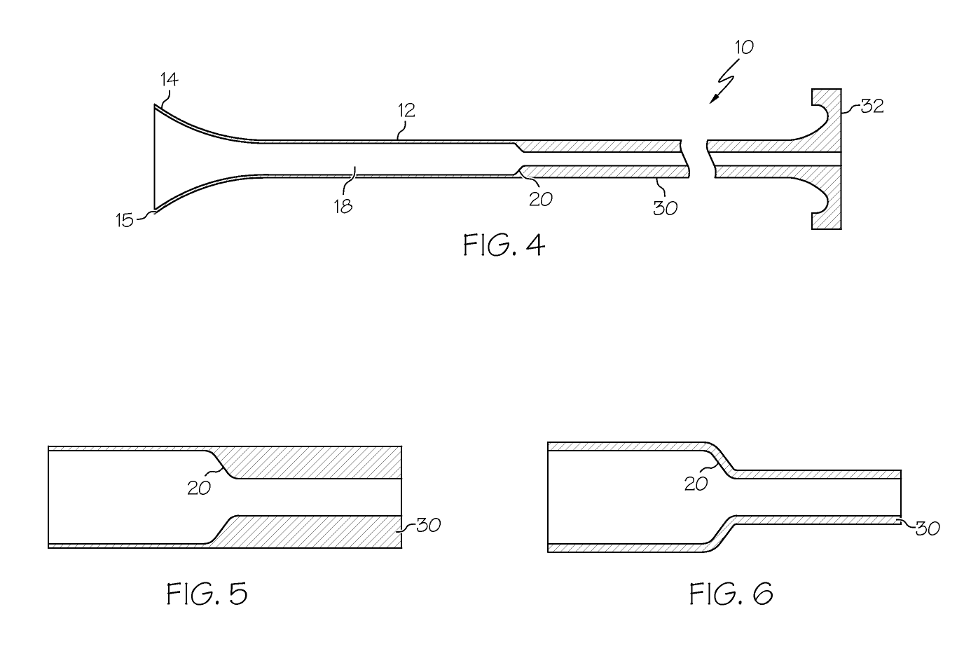

FIGS. 1 and 4 show cross-sectional views of an embodiment of a recapturing sheath 10 that can be used in conjunction with a drug delivering medical device, such as an inflation balloon. Desirably, the recapturing sheath 10 comprises a sheath portion 12 having a distal end 15 that comprises a flare 14 (see FIG. 4). The sheath portion 12 is desirably tubular, thus defining an internal lumen 18, and arranged to surround a drug delivering medical device.

In some embodiments, the recapturing sheath 10 is provided without a drug delivering medical device. Desirably, a drug delivering medical device, such as a balloon having a drug coating, will be positioned in the internal lumen 18, and the balloon and recapturing sheath 10 can be advanced to a treatment site.

In some embodiments, the recapturing sheath 10 is provided with a drug delivering medical device already oriented within the internal lumen 18. In some embodiments, the entire assembly is provided in a sealed, sterilized package.

In some embodiments, the recapturing sheath 10 comprises a balloon seat 20, for example located at a proximal end of the sheath portion 12. In some embodiments, the balloon seat 20 comprises a reduction in cross-sectional area of an internal lumen 18 defined by the recapturing sheath 10. A balloon oriented within the lumen 18 can abut the seat 20. In embodiments that comprise a seat 20, a balloon is desirably loaded into the recapturing sheath 10 from the distal end 15. In embodiments that do not comprise a seat, a balloon can be inserted from a proximal end of the lumen 18.

In some embodiments, the recapturing sheath 10 comprises an elongate shaft portion 30. In some embodiments, the recapturing sheath 10 further comprises a backstop 32. The internal lumen 18 can extend through the elongate shaft portion 30 and backstop 32.

In some embodiments, the recapturing sheath 10 is configured as an over-the-wire device, wherein the recapturing sheath 10 can be oriented around a guidewire (not shown), and the guidewire can be used to guide the recapturing sheath 10 to a treatment site.

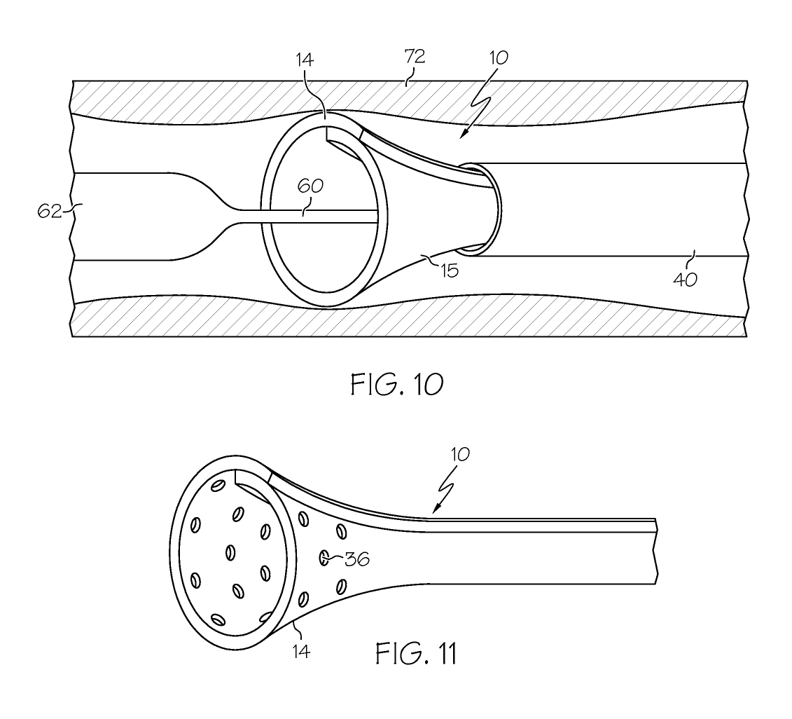

In some embodiments, the recapturing sheath 10 is configured for use with a guide catheter 40 (see FIG. 10), wherein the recapturing sheath 10 can be placed within a lumen of a guide catheter 40, and the guide catheter 40 can be used to guide the recapturing sheath 10 to a treatment site.

In some embodiments, the recapturing sheath 10 comprises an insertion sheath 50. FIGS. 1-3 illustrate an embodiment of an insertion sheath 50. Desirably, an insertion sheath 50 will protect the recapturing sheath 10 from damage or contamination when inserting the recapturing sheath 10 into a guiding device, such as a guide catheter 40. An insertion sheath 50 can be made from any suitable material, such as a polymer.

An insertion sheath 50 can have any suitable length. Desirably, an insertion sheath 50 will cover at least a portion of the distal end of the sheath portion 12. In some embodiments, an insertion sheath 50 will cover a greater amount of the sheath portion 12 and may extend to a stop 20, or beyond a stop 20.

Desirably, the insertion sheath 50 is configured to be removable. In some embodiments, the insertion sheath 50 comprises a handle 52 that allows an operator to grasp a portion of the insertion sheath 50 more easily. In some embodiments, the insertion sheath 50 is arranged to split in a predetermined configuration, for example splitting along a line of perforations. In some embodiments, the handle 52 is also arranged to split. For example, the handle 52 may be split into two portions, wherein a first portion of the handle 52 is attached to a first portion of the insertion sheath 50 material, and a second portion of the handle 52 is attached to a second portion of the insertion sheath 50 material. FIG. 3 illustrates a handle 52 being split.

When used with a guide catheter 40, a recapturing sheath 10 is desirably oriented with its distal end 15 placed within the guide catheter 40. The insertion sheath 50 protects the recapturing sheath 10 during insertion. The insertion sheath 50 can then be removed. In some embodiments, a lubricant can be used in conjunction with the insertion sheath 50 to reduce friction. For example, a lubricant can be placed on an inner surface of the insertion sheath 50, to reduce frictional engagement with the sheath portion 12 of the recapturing sheath 10. A lubricant can be placed on an outer surface of the insertion sheath 50, to reduce frictional engagement with an object that may surround the insertion sheath 50, such as a guide catheter 40.

FIG. 1 shows an embodiment of a recapturing sheath 10 with the flared end portion 15 in a reduced delivery configuration and contained within the insertion sheath 50.

FIG. 4 shows an embodiment of a recapturing sheath 10 in a flared configuration (e.g. expanded/unconstricted), wherein the flared distal end 15 assumes a larger size. Desirably, a perimeter of the sheath portion 12 at the flare 14 is greater than a perimeter of the sheath portion 12 at locations spaced away from the distal end 15. Desirably, the size of the flare 14, when unconstricted, continually increases toward the distal end 15.

FIG. 5 shows an embodiment of a recapturing sheath 10 having a seat 20, wherein an outer diameter of the recapturing sheath 10 is constant. The seat 20 is defined by a reduction in size of the internal lumen. A wall thickness of the recapturing sheath 10 increases at the seat 20, and the thicker wall continues along the elongate shaft portion 30.

FIG. 6 shows an embodiment of a recapturing sheath 10 having a seat 20, wherein a wall thickness of the recapturing sheath 10 is constant across the seat 20. The outer diameter of the recapturing sheath 10 is reduced at the seat 20, and the elongate shaft portion 30 continues having the reduced size.

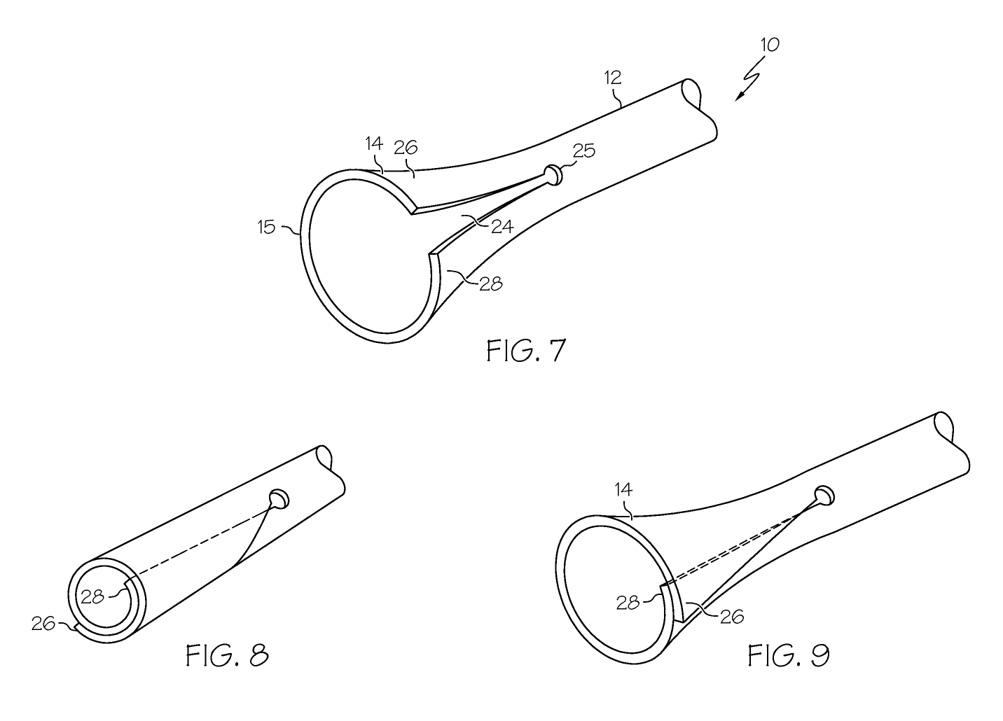

FIGS. 7-9 show a distal end of an embodiment of a recapturing sheath 10. In some embodiments, the distal end 15 of the sheath portion 12 comprises a slit 24. A first portion 26 of material is defined on one side of the slit 24, and a second portion 28 of material is defined on the other side of the slit 24. The first portion 26 is configured to overlap the second portion 28, for example as shown in FIGS. 8 and 9. The amount of flaring of the distal end 15 can be adjusted by adjusting the amount of overlap.

FIG. 8 shows a reduced configuration, wherein the distal end 15 flares less and includes a greater amount of overlapped area. In some embodiments, an outer diameter of the distal end 15 in the reduced configuration is equal to or less than an outer diameter of the elongate shaft portion 30. In some embodiments, an outer diameter of the distal end 15 in the reduced configuration is equal to or less than an outer diameter of the sheath portion 12 at a location spaced away from the distal end 15.

FIG. 9 shows a configuration that is more flared than the configuration of FIG. 8. The amount of area overlapped by the first portion 26 has been reduced, and the size of the flare 14 has increased.

In some embodiments, the recapturing sheath 10 is designed to assume a more flared configuration when at-rest. Thus, the recapturing sheath can be designed to assume the configuration illustrated in FIG. 9, or in some embodiments, the configuration of FIG. 7, when at rest. The recapturing sheath 10 can be reduced to the configuration of FIG. 8 and placed within a constriction device, such as an insertion sheath 50. Desirably, the internal forces of the recapturing sheath 10 cause the flared end portion to assume a larger flared size when the constriction device is removed.

In some embodiment, a proximal end of the slit 24 comprises an enlargement 25. An enlargement 25 helps prevent the proximal end of the slit 24 from binding or otherwise causing difficulty in the overlap of the first portion 26 and second portion 28.

FIG. 10 illustrates an embodiment of a recapturing sheath 10 in use, oriented within a vessel 72. The recapturing sheath 10 and a catheter 60 with a drug delivery balloon 62 were advanced to the treatment site within a guide catheter 40. At the treatment site, the flare 14 of the recapturing sheath 10 has been advanced out of the guide catheter 40. Desirably, the recapturing sheath 10 flares to a larger size when no longer constricted by the guide catheter 40. Desirably, an expanded diameter of the flare 14 exceeds an outer diameter of the guide catheter 40. In some embodiments, an expanded diameter of the flare 14 is at least 1.25 times the outer diameter of the guide catheter 40. In some embodiments, an expanded diameter of the flare 14 is at least twice the outer diameter of the guide catheter 40. In some embodiments, an outer surface of the flare 14 contacts the vessel 72. The drug delivery balloon 62 has been advanced out of the recapturing sheath 10, and is delivering drugs to the treatment site, such as a portion of the vessel 72. In some orientations, for example when the recapturing sheath 10 is located downstream from the drug delivery balloon 62, the flare 14 can capture drugs that may be lost in the bloodstream and prevent the drugs from being released from the proximity of the treatment site.

Once the drug treatment is completed, the drug delivery balloon 62 is deflated and withdrawn into the recapturing sheath 10. As the drug delivery balloon 62 passes into the guide catheter 40, the recapturing sheath 10 helps to prevent drug coatings from being removed from the drug delivery balloon 62. For example, the recapturing sheath 10 can prevent a distal edge of the guide catheter from scraping drug coating off the balloon 62. Desirably, the recapturing sheath 10 is left in the advanced and flared configuration until after the drug delivery balloon 62 is withdrawn completely into at least the flare 14. Thus, even if the act of withdrawing the balloon 62 into the guide catheter 40 does remove drug coating, the removed drug coating will be contained within the recapturing sheath 10. The recapturing sheath 10 and balloon 62 can be fully withdrawn, and the entire assembly can be removed from the vessel 72.

Desirably, the distal end 15 and flare 14 of the recapturing sheath 10 comprise an atraumatic material that will not damage a vessel wall 72.

The distal end 15 and flare 14 are desirably be made of a thin material. In some embodiments, a wall thickness of the sheath portion 12 can range from less than 0.001″ to 0.02″ or greater. In some embodiments, a wall thickness of the sheath portion 12 changes, and is thinner at the flare 14. In some embodiments, a wall thickness of the sheath portion 12 is constant.

In some embodiments, the flare 14 comprises a material that is softer or more elastic than the rest of the recapturing sheath 10. In some embodiments, the sheath portion 12 comprises a material that is softer or more elastic than the rest of the recapturing sheath 10 (e.g. than the elongate shaft portion 30).

In some embodiments, the elongate shaft portion 30 can be made of any suitable material, such as catheter materials including, but not limited to, moldable polymers, polyether block amide (PEBA), nylon or polyethyleneterephthalate (PET), polyurethane, latex, silicone rubber, natural rubber, polyvinyl chloride, polyamide, polyamide elastomer, copolymer of ethylene and vinyl acetate, polyethylene, polyimide, stainless steel and suitable alloy materials such as nickel-titanium alloys, cobalt-chromium-nickel alloys, etc. In some embodiments, the elongate shaft portion 30 comprises a material having a durometer ranging from 20 D to 80 D. In some embodiments, the elongate shaft portion 30 comprises a material having a durometer ranging from 50 D to 70 D.

In some embodiments, at least the distal end 15 of the sheath portion 12 comprises a soft material having a durometer value in the range of 20 A to 60 A. In some embodiments, at least the distal end 15 of a sheath portion 12 that includes a slit 24 (see FIG. 7) comprises a durometer value in the range of 35 A to 60 A. In some embodiments, at least the distal end 15 of a sheath portion 12 that does not include a slit 24 comprises a durometer value in the range of 20 A to 45 A, thus allowing for better foldability.

In some embodiments, the entire recapturing sheath 10 is made from a single material. Desirably, the elongate shaft portion 30 is thicker than the distal end 15/flare 14, allowing the elongate shaft portion 30 to be stronger than the distal end 15.

The recapturing sheath 10 may be made using any suitable method, such as extrusion, bump extrusion, molding, heat forming, etc. In some embodiments, the recapturing sheath 10 is formed with a flare 14. In some embodiments, the recapturing sheath 10 can be formed without a flare 14 (e.g. tubular extrusion of constant diameter), and the flare 14 can be imparted to the distal end, for example by mechanical flaring. In some embodiments, for example when the elongate shaft portion 30 comprises a first material and the flare 14 comprises a second material, the elongate shaft portion 30 and the flare 14 can be formed independently, then attached to one another. Any suitable attachment method can be used, such as an adhesive, suitable bonding/welding techniques such as heat welding, sonic welding and other suitable coalescence techniques, etc.

FIG. 11 shows an embodiment of a recapturing sheath 10 wherein the flare 14 comprises a plurality of apertures 36. Each aperture 36 extends through the thickness of the wall of the flare 14. In some embodiments, the apertures 36 can extend proximal to the flare 14, along a greater length of the sheath portion 12. Apertures 36 may have any suitable size and shape, and multiple apertures 30 may be arranged in any suitable pattern. In some embodiments, the flare 14 comprises a plurality of apertures 36 spaced around a circumference of the flare 14. In some embodiments, the flare 14 further comprises a second plurality of apertures 36 spaced around a second circumference of the flare 14. In some embodiments, the first plurality of apertures 36 is rotationally staggered with respect to the second plurality of apertures 36.

In various embodiments, a recapturing sheath 10 having apertures can be configured to allow for perfusion, filtration, etc. In some embodiments, a distal end of the recapturing sheath 10 comprises a filter.

When the recapturing sheath 10 of FIG. 11 is oriented within a vessel, the apertures 36 allow some amount of blood flow through the wall of the flare 14 and thus around/through the recapturing sheath 10.

In some embodiments, the distal end 15 of the recapturing sheath 10 does not include a slit 24 (see FIG. 7). Desirably, the distal end 15 of such embodiments is folded to achieve a reduced delivery configuration.

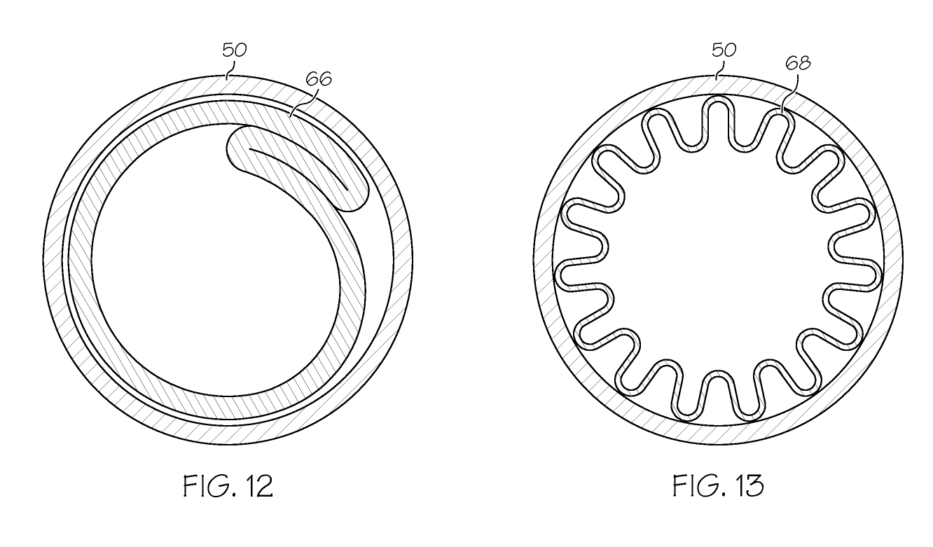

FIGS. 12 and 13 each show a cross-sectional view of an embodiment of a flare that is folded to a reduced size and contained within an insertion sheath 50. FIG. 12 shows radial folds 66, wherein portions of the flare overlap one another in a radial direction. FIG. 13 shows circumferential folds 68, wherein portions of the flare overlap one another in a circumferential direction.

In some embodiments, a recapturing sheath 10 can be used with any suitable drug delivering medical device, not just balloons.

In some embodiments, a recapturing sheath 10 can be used with a stent. In some embodiments, a stent can be disposed about an inflation balloon and delivered to a treatment site similar to the balloon 62 described with respect to FIG. 10.

This invention is also directed to methods of making and using the various embodiments of a recapturing sheath 10 as disclosed herein.

A recapturing sheath 10 can be used to contain any suitable drugs and/or drug coatings, for example any drug that may be present on an intravascular medical device. Such coatings can include one or more non-genetic therapeutic agents, genetic materials and cells and combinations thereof.

Non-genetic therapeutic agents include anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, endostatin, angiostatin and thymidine kinase inhibitors; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides; vascular cell growth promoters such as growth factor inhibitors, growth factor receptor antagonists, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vascoactive mechanisms.

Genetic materials include anti-sense DNA and RNA, DNA coding for, anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor .alpha. and .beta., platelet-derived endothelial growth factor, platelet-derived growth factor, tumor necrosis factor .alpha., hepatocyte growth factor and insulin like growth factor, cell cycle inhibitors including CD inhibitors, thymidine kinase (“TK”) and other agents useful for interfering with cell proliferation the family of bone morphogenic proteins (“BMP's”), BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Desirable BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

Cells can be of human origin (autologous or allogeneic) or from an animal source (xenogeneic), genetically engineered if desired to deliver proteins of interest at the transplant site. The cells may be provided in a delivery media. The delivery media may be formulated as needed to maintain cell function and viability.

Polymer coating materials can include polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof, coatings from polymer dispersions such as polyurethane dispersions (for example, BAYHDROL®), fibrin, collagen and derivatives thereof, polysaccharides such as celluloses, starches, dextrans, alginates and derivatives, hyaluronic acid, squalene emulsions. Polyacrylic acid, available as HYDROPLUS® (Boston Scientific Corporation, Natick, Mass.), and described in U.S. Pat. No. 5,091,205, the disclosure of which is hereby incorporated herein by reference, is particularly desirable. Even more desirable is a copolymer of polylactic acid and polycaprolactone.

The above disclosure is intended to be illustrative and not exhaustive. This description will suggest many variations and alternatives to one of ordinary skill in this field of art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. A recapturing sheath comprising:
an elongate shaft having a proximal end and a distal end, said shaft defining a lumen therein;
said distal end comprising a flare and a slit, wherein a first portion of said flare overlaps a second portion of said flare.

2. The recapturing sheath of claim 1, wherein a size of said flare is adjustable by changing an amount of said overlap.

3. The recapturing sheath of claim 1, wherein a perimeter of material at said distal end is greater than a perimeter of material proximal to said slit.

4. The recapturing sheath of claim 1, wherein said lumen comprises an internal seat.

5. The recapturing sheath of claim 4, wherein said seat comprises an increase in thickness of a wall of said elongate shaft.

6. The recapturing sheath of claim 4, wherein said seat comprises a contour in a wall of said elongate shaft portion, said wall having a constant thickness.

7. The recapturing sheath of claim 1, wherein said flare comprises a plurality of apertures.

8. The recapturing sheath of claim 1, further comprising an insertion sheath surrounding at least said distal end.

9. The recapturing sheath of claim 1, wherein said flare comprises a softer material than said elongate shaft.

10. An assembly comprising:
a drug delivering medical device; and
a recapturing device comprising an elongate shaft portion and a distal sheath portion, said distal sheath portion comprising a flared distal end, said recapturing device having an internal lumen, said medical device oriented within said lumen; and
an insertion sheath surrounding at least a distal portion of said recapturing device.

11. The assembly of claim 10, the internal lumen of the recapturing device comprising a seat portion.

12. The assembly of claim 11, wherein said seat portion comprises a reduction in a cross-sectional area of said lumen.

13. The assembly of claim 10, said flared distal end comprising a slit, wherein a first portion of said flared distal end overlaps a second portion of said flared distal end.

14. The assembly of claim 13, wherein a proximal end of said slit comprises an enlargement.

15. The assembly of claim 10, wherein said flared distal end comprises a plurality of apertures.

16. The assembly of claim 10, further comprising a guide catheter, said recapturing device being longer than said guide catheter.

17. The recapturing sheath of claim 10, wherein said flared distal end comprises a softer material than said elongate shaft portion.

18. The recapturing sheath of claim 10, wherein said flared distal end comprises at least one radial fold.

19. The recapturing sheath of claim 10, wherein said flared distal end comprises at least one circumferential fold.

20. A method comprising:
providing a drug delivering medical device and a recapturing device comprising a flared end portion;
delivering said medical device and said recapturing device to a treatment site;
orienting the flared end portion in a flared configuration;
delivering said drug to the treatment site while containing at least some of said drug in said recapturing device.
